(19) United States
(12) Plant Patent Application Publication
(10) Pub. No.: US 2009/0019611 P1
(43) Pub. Date: Jan. 15, 2009

(54) Delta Shade

(76) Inventors: Stacie A. Zinn, Naples, FL (US); Rex Cunningham, Avon Park, FL (US); Wiley T. McCall, Avon Park, FL (US); Laurie E. Trenholm, Melrose, FL (US); Ralph White, Tavares, FL (US)

Correspondence Address: STACIE ZINN, 1765 COVRTYARD WAY #101, NAPLES, FL, 34112, US

(21) Appl. No.: 11/827,496

(22) Filed: Jul. 13, 2007

(51) Int. Cl. A01H 5/00

(52) U.S. Cl. PLT/392

(57) ABSTRACT

DeltaShade is an attractive perennial, vegetatively propagated Stenotaphrum secundatum (St. Augustine grass) having a bright green color, shade tolerance, cold tolerance and a semi-dwarf leaf blade.

### GENUS AND SPECIES NAME

The Latin name of the genus and species of the novel variety herein is Stenotaphrum secundatum.

Variety denomination: The inventive variety of St. Augustine grass disclosed herein has been given the variety denomination DeltaShade.

### BACKGROUND OF THE INVENTION

Stenotaphrum secundatum belongs to the grass family, Poaceae, and has the common names St. Augustine and buffalo grass. Stenotaphrum secundatum is a vigorous-growing perennial grass that prefers warm weather climates and is able to withstand temperatures as high as 105 degrees F. Its market class is turf grass, and it is widely used as a lawn grass in the Southern United States where many other grasses cannot survive the extreme temperatures.

Some commonly known varieties of S. secundatum include ‘Floratine’ (unpatented), ‘Bitter Blue’ (unpatented), ‘SS-100’ (U.S. Plant Pat. No. 9,395; sold under the name PALMETTO™), and ‘Sir Walter’ (Australian PBR No. 96/226; unpatented in the United States), and others noted in the Citation Section.

The variety known as DeltaShade is a naturally occurring selection of St. Augustine discovered, isolated and propagated in Tavares, Fla. in the late 1980s. The maternal plant was then split into stolons and taken to test sites in Florida and Central Tennessee where it was again isolated and propagated under differing weather conditions. It has been observed and tested in both states since its discovery.

DeltaShade is a distinct, asexually propagated variety of St. Augustine grass. The plant is so identified in portions of the drawings of this disclosure.

### SUMMARY OF INVENTION

DeltaShade is a distinctive, bright green St. Augustine grass exhibiting shade tolerance, cold tolerance and a semi-dwarf leaf length.

### BRIEF DESCRIPTION OF THE DRAWINGS

FIG. 1: This close-up photo of DeltaShade St. Augustine shows the distinctive bright green color and semi-dwarf leaf texture of the grass.

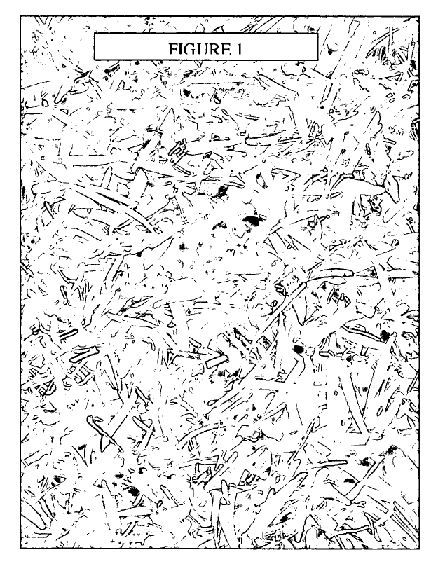

FIG. 2: This photo shows an example of the types of structures used by the University of Florida researchers for shade tolerance studies in their greenhouse. Foreground are full sun treatments, left rear is 30% shade treatment. Right rear is 70% shade treatment.

FIG. 3: DeltaShade is shown to retain strong turf quality at 30% shade, providing good shade tolerance, just behind scores for Amerishade.

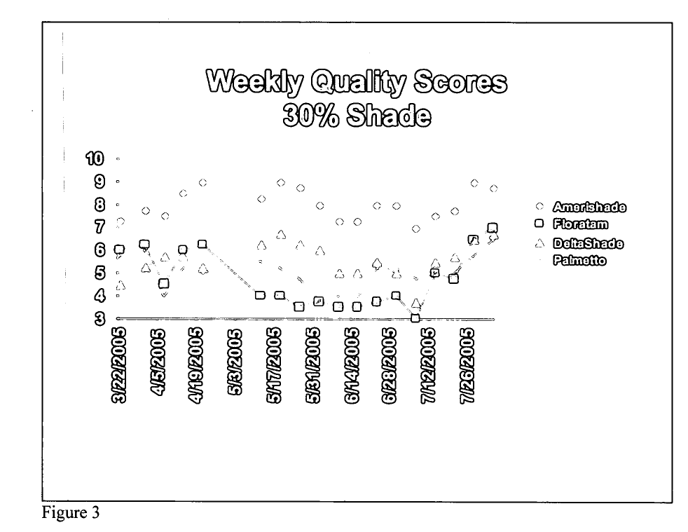

FIG. 4: DeltaShade is shown to retain strong turf quality at 50% shade, providing good shade tolerance, just behind scores for Amerishade.

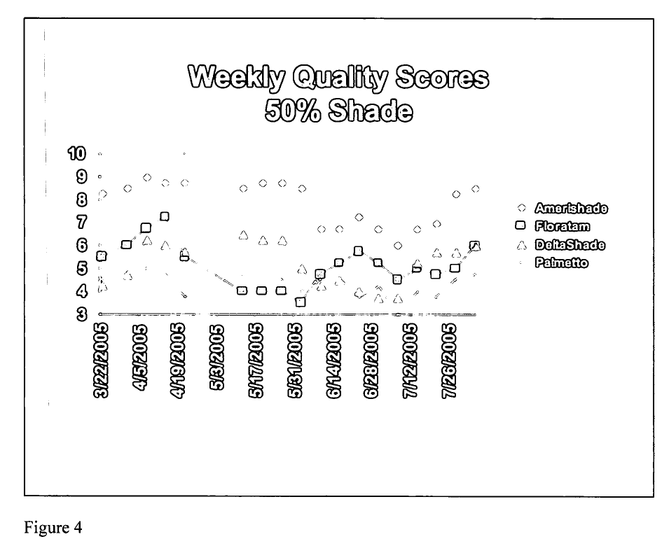

FIG. 5: DeltaShade is show to retain strong turf quality at 70% shade, providing good shade tolerance, just behind scores for Amerishade.

FIG. 6: Seasonal Density tests also show strong results from DeltaShade when tested against Amerishade, Floratam and Palmetto for Seasonal Density at 70% shade.

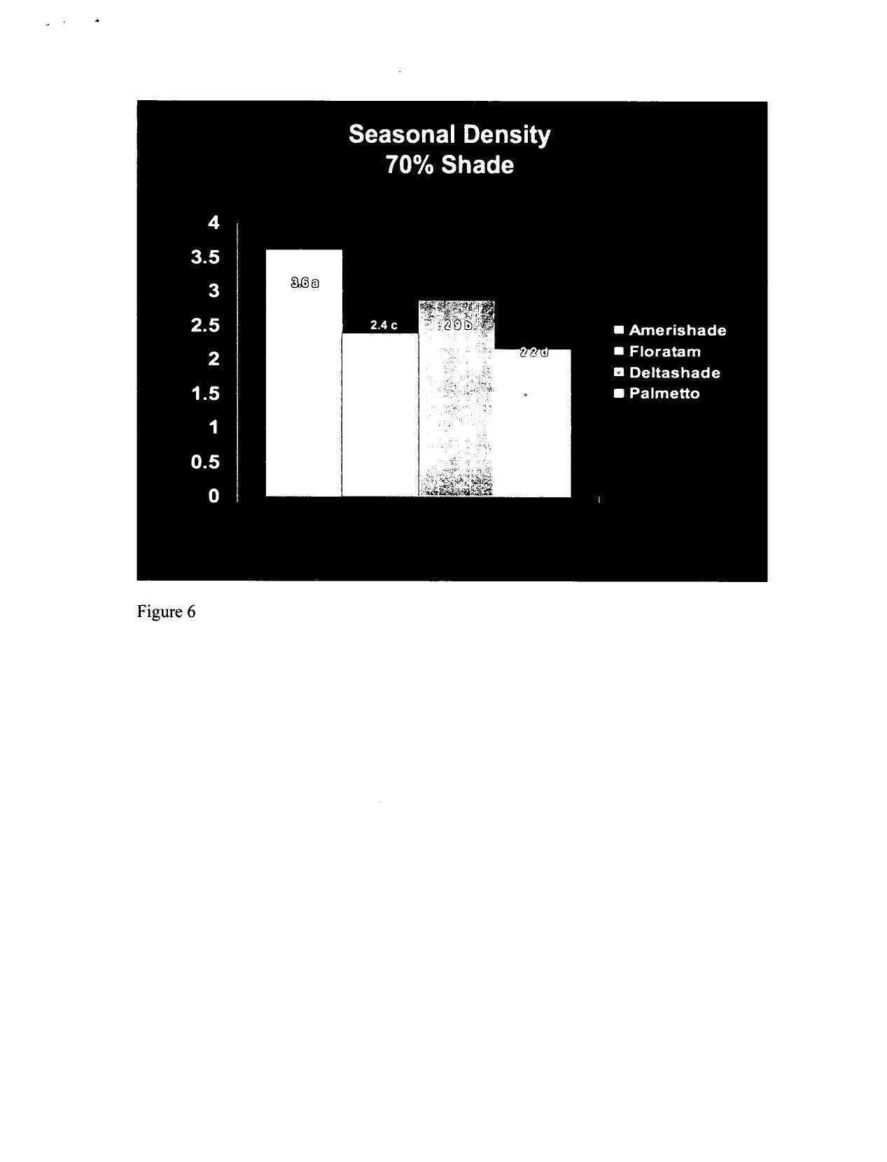

### DETAILED BOTANICAL DESCRIPTION OF THE VARIETY

The following is a detailed description of a new Stenotaphrum secundatum grass variety known as DeltaShade. The description is based on observations of the plant grown nursery field plots and greenhouses.

DeltaShade is a perennial, vegetatively propagated grass discovered and isolated, then vegatatively propagated. It grows by creeping or ascendanta stolons which root at the nodes. Over repeated propagations, ‘DeltaShade’ has remained genetically stable.

Basic Characteristics

DeltaShade is a unique variety possessing both cold tolerance and shade tolerance.
Winter Hardiness/Cold Tolerance: Tested as far north as Tennessee.
Shade Tolerance: Tested up to maximum of 70% shade.
Growth Habit: Prostrate, low and spreading
Color: Apple green color at low levels of fertilization
Drought Tolerance: Good. Attributed to its deep root system
Soil Type: When adequately fertilized, appears indifferent to soil type
Disease: No serious disease infestation observed at this time
Insects: No chinch bug infestation observed in more than a decade.
   Range of Adaptation

The environmental range of adaptation is not fully known. DeltaShade St. Augustine should perform well in Southeastern United States and Southern California.

Test Sites

Jacksonville, Fla.: Since 1995, DeltaShade has been planted in heavy shade under oak trees on a residential lawn and continues to thrive.

Tavares, Fla.: Since 1995, DeltaShade has been planted in heavy shade on residential lawn and continues to thrive.

Central Tenn.: From 1998 to 2004, DeltaShade was planted on a sod farm under constant professional supervision and continued to thrive.

Bay St. Louis, Miss.: From November 2004 until the destruction of the college test site caused by Hurricane Katrina, DeltaShade was planted under a tree and supervised by a sports turf management professional at St. Stanislaus College. On Christmas Day of December 2004, the area around St. Stanislaus College received snowfall and yet the DeltaShade planted there remained green. (Study continued until Hurricane Katrina destroyed the college in August 2005. Sample plots could not be located in the devastation.)

Gainesville, Fla.: Since 2005, DeltaShade has been at the University of Florida greenhouses undergoing Shade Tolerance studies.

Technical Description and Test Results

1. The overall color of DELTASHADE is a bright green color. The color is classified as a range between 5GY (4/6 to 5/6) to 7.5GY (4/6 to 5/6) on the Munsell color chart.

2. The average leaf blade length and width (in mm) is as follows:

| | | |
|---|---|---|
| First expanded: | length 31 +/− 16 | width 7.4 +/− 1.6 |
| Third expanded: | length 3 +/− 12 | width 8.3 +/− 1.7 |

note: leaf width taken one have distant from collar to tip of blade

3. Internode length and width (in mm) is as follows:

| | | |
|---|---|---|
| First internode: | length 57 +/− 4 | width 3.25 +/− 75 |
| Third internode: | length 60.75 +/− 2.25 | width 3.5 +/− .5 |

4. Inflorescence (in mm) is as follows:
Spikelets 4.6 mm average
Length 106 mm, average 97.6 mm

5. Color of Anthers: orange-yellow

6. Color of Stigma: white

7. Average height of unclipped grass: 12-14-inches

8. Chromosome number: 18

9. Growth habit: Prostrate, low and spreading

10. Cold tolerance:

The following is a synoptic history of DeltaShade St. Augustine that was in tests from June of 1998 to June 2004 at Charles Williams & Associates, Inc., turf farms in Fayetteville, Tenn.

DeltaShade was planted in a 5′×5′ plot in Central Tennessee in 1998. There was a preplant fertilizer applied and one subsequent application of ammonium nitrate applied approximately 14 days later. The planting was done by washing out the pieces of sod and then cutting the runners up into pieces, each having 3 nodes and planting them in the plot which had been worked by hand. Establishment rate was excellent, with the area being fully covered between 8 and 9 weeks.

During the six years that DeltaShade was maintained and observed in the plot, the area climatic conditions covered a wide range of extremes. The mean average normal temps range from a low of 28° in January to highs of 90° plus in July and August. The lowest temps during the six year observation period −1°, 0°, and 1° with wind chill factors in the −20° range. In that time, the test facility had one period of snow cover of 6 to 8 inches that lasted for 7 days and several others of 1 to 4 inches that lasted 1 to 5 days. The highest temps were experienced during the drought of 1999 during which temps for one 3-week period ran between 95° and 101°.

11. Shade Tolerance:

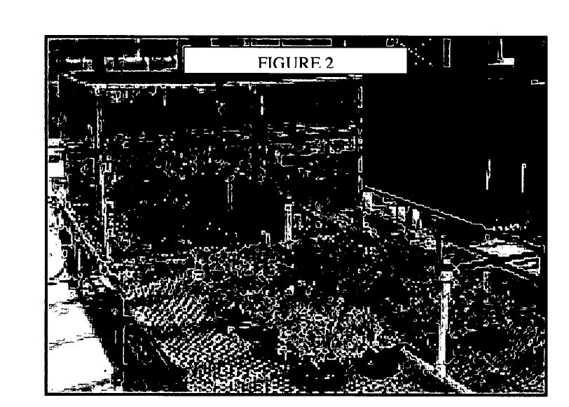

The following is a summary of the shade tolerance tests conducted on DeltaShade and three other St. Augustine varieties known as: Amerishade, Floratam and Palmetto. The tests were conducted at the University of Florida in Gainesville in replicated tests. The grasses were subjected to 4 different light regimes (0, 30, 50, and 70% shade). Grasses were grown in 6-inch pots placed under PVC-frame shade structures fitted with shade cloth to provide 30, 50, or 70% shade (FIG. 2 ).

In preliminary studies of all four grasses, DeltaShade tested better in up to 70 percent shade than Floratam and Palmetto in weekly quality shade scores. Only AmeriShade had better shade tolerance.

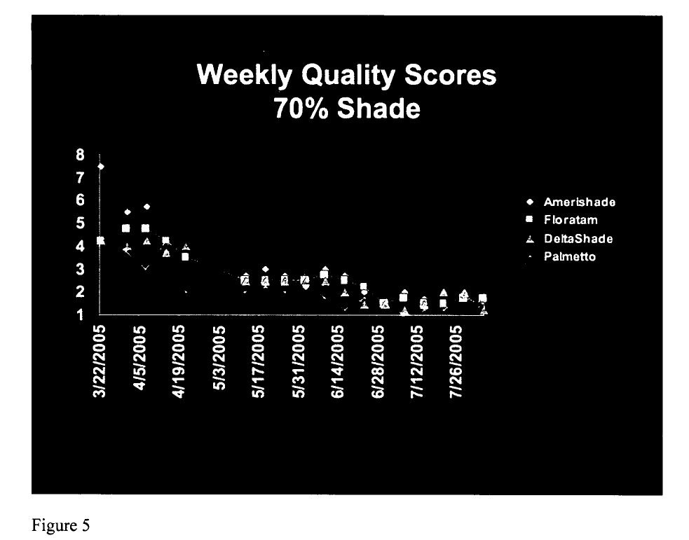

Seasonal Density tests also show strong results from DeltaShade when tested against Amerishade, Floratam and Palmetto for Seasonal Density at 70% shade. See FIG. 5 below.

In addition, visual observations at the Central Tennessee test site yielded the following: Tests under a tree: Shade tolerance rated as “good.”

In other industry tests not sited here, AmeriShade has been shown to have little cold tolerance. These results are significant in that DeltaShade's cold tolerance (as submitted in section 10) coupled with its shade tolerance (demonstrated here in section 11) make DeltaShade a unique variety possessing both cold and shade tolerance.

What the claim is:

1. A new and distinct variety of Stenotaphrum secundatum plant named DeltaShade, with

2. cold tolerance,

3. shade tolerance,

4. bright green color and

5. semi-dwarf leaf size, substantially as illustrated and described herein.
